(19) United States
(12) Patent Application Publication
(10) Pub. No.: US 2011/0132792 A1
(43) Pub. Date: Jun. 9, 2011

(54) MEDITOWEL PRE-PACKAGED MEDICATED ILLUMINATING SKIN BRIGHTENER WIPE

(76) Inventor: Sally Villalobos, Montebello, CA (US)

(21) Appl. No.: 12/963,897

(22) Filed: Dec. 9, 2010

Related U.S. Application Data
(60) Provisional application No. 61/285,113, filed on Dec. 9, 2009.

(51) Int. Cl. B65D 85/00; A61K 8/34; A61Q 19/02

(52) U.S. Cl. 206/524.1; 424/62

(57) ABSTRACT

Single use, substrate uniformly impregnated with an anhydrous illuminating skin brightener solution for the use of aseptically delivering a single dose of an illuminating skin brightener solution to the area of discoloration. Solution and substrate is contained encapsulated within a heat sealed foil enclosure that is stable for at least five years.

### CROSS-REFERENCE TO RELATED APPLICATION

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATION-BY-REFERENCE OF MATERIAL SUBMITTED ON A COMPACT DISC

Not applicable

### BACKGROUND OF THE INVENTION

1. Field of the Invention

The invention relates to a medicated illuminating skin brightener wipes. More particularly, it relates to a pre-packaged single use disposable towelette/wipe that contains a full dose of a skin brightening solution.

2. Description of Related Art Including Information Disclosed Under 37 CFR 1.97

The market is saturated with skin whitener creams and ointments. These products are packaged in many types of bottles and tubes, but none provide the portability and effectiveness of our invention.

Hydroquinone, the main component to this composition is found in a number of patents related to skin brighteners: U.S. Pat. No. 4,466,955 to Calvo, et al. (Skin bleaching stick containing Hydroquinone); U.S. Pat. App. No. 11/290,966 to Rubin (Skin care composition comprising hydroquinone and a substantially anhydrous base); U.S. Pat. No. 4,692,261 to Filomeno (Skin bleaching detergent bar); this invention builds upon these prior patents and a novel and unique product is created that provides portable and potent skin illumination.

The application is for an individually wrapped medicated illuminating skin brightener wipe. The use of the product as a portable wipe differs from other products. Instead of a bottle or tube that must be reused over and over, this product coats the area with a single dose of the skin illuminating composition at an over-the-counter (“OTC”) level. Using a wipe is far more efficient and portable. With a tube or bottle, the dosage is eye balled and too much or too little may be used. The use of a bottle or tube also allows for the contamination of the bottle. Once the bottle or tube is open, the product is exposed to air and particles which may contaminate the cream inside their enclosure.

Each wipe is individually enclosed in an impermeable package that can maintain the wipes potency for at least 5 years. This allows the user to quickly administer the medication anywhere without having the nuisance of carrying a tube or bottle.

The novel concept of the skin illuminating wipe is rooted in the proprietary medicated composition. The main compositions consist of Hydroquinone, Propylene Glycol, Glycerin, Arnica extract, Sodium Metabisulfate, Allantoin, Bisabolol and Aloe Vera Gel.

The Key ingredient to brightening and whitening of the skin is Hydroquinone. Hydroquinone is a type of phenol and is considered an aromatic organic compound that has the chemical formula of C₆H₄(OH)₂. In cosmetics it is considered a skin bleaching agent that can reduce the intensity of certain skin hyper pigmentations or hypermelanosis such as freckles, melasma also known as “mothers marks” due to its occurrence in pregnant woman, and senile lentigines commonly known as “liver spots”. When topical products containing hydroquinone are applied to the skin over a period of time, the skin becomes lighter.

The remaining additives are important for healing the skin while it is becoming lighter and brighter.

Glycerin aids in moisturizing and coating the compromised skin keeping it hydrated while it heals. Glycerin further helps to restore the healthy look and feel to the skin without introducing water which may dehydrate too quickly and not give the healing ingredients time to penetrate. Glycerin has been used for a number years in cosmetic patents. U.S. Pat. No. 7,361,363 to Barrow, et al. (Silky feel cosmetic emulsion chassis based on glycerin and chemically modified starch); U.S. Pat. No. 6,162,775 to Mathmanus-Spaltro (Anhydrous Liquid Cosmetic Composition Comprising Glycerin and Polyalkylene Glycol).

Aloe Vera for centuries has been known for its natural skin healing properties. It is known for naturally soothing the skin, promoting healing and cellular renewal.

Arnica extract is traditionally known for its effect on reducing bruising of impacted skin. Arnica has been demonstrated to act as an anti-inflammatory and assist normal healing processes by facilitating transport of blood and fluid accumulations through a dilating action of subcutaneous blood capillaries.

Allantoin is a natural ingredient obtained from the comfrey plant, known for its gentleness on the skin. It is a very healing, soothing and anti-irritating ingredient. Used in this invention for its superb ability to protect the skin while it heals. Allantoin also has anti-inflammatory properties.

Bisabolol is another ingredient that is known for its skin healing effects, aids in reducing scars, acts as an anti-irritant and anti-inflammatory. Since Hydroquinone has a low pH, it can be irritating to the skin therefore skin healing ingredients were added to help the skin through the process of lightening.

Sodium Metabisulfate, Methylparaben and Propylparabens aid in preserving the substrate.

The medicated Illuminating Skin brightener Wipe is enclosed in an impermeable package that can maintain the wipes potency for at least 5 years.

The following listed patents further illustrate less relevant pre-packaged sheet materials saturated with medicaments: U.S. Pat. No. 2,402,982 to Steenbergen (bandage in petrolatum): U.S. Pat. No. 2,999,265 to Duane et al. (hermetically sealed pad impregnated with epidermal bacteriostatic agent, humectant and emollient): U.S. Pat. No. 3,783,869 to Schnipper (medicated sanitary napkin).

Although a patent have been granted for pain relief compositions using similar and some of the same components, the utility of having an aseptic pre-packaged portable wipe that can be used to apply the topical composition is novel.

### BRIEF SUMMARY OF THE INVENTION

The present invention is focused on a pre-packaged medicated illuminating skin brightener wipe. The wipe is manufactured as a substrate uniformly impregnated with medicated solution. Stability studies show that the substrate, which as a product, is enclosed in a package, will hold the wipes potency and moisture for more than 5 years. In use, the towelette/wipe is a portable single use towelette that provides the user with a single dose of the illuminating skin brightener solution to the skin to remove darkened spots and discolorations, while simultaneously refining, hydrating and moisturizing the texture of the skin. After treatment the wipe is discarded.

### BRIEF DESCRIPTION OF THE DRAWINGS

FIG. 1 is a perspective view of the package enclosing the wipes

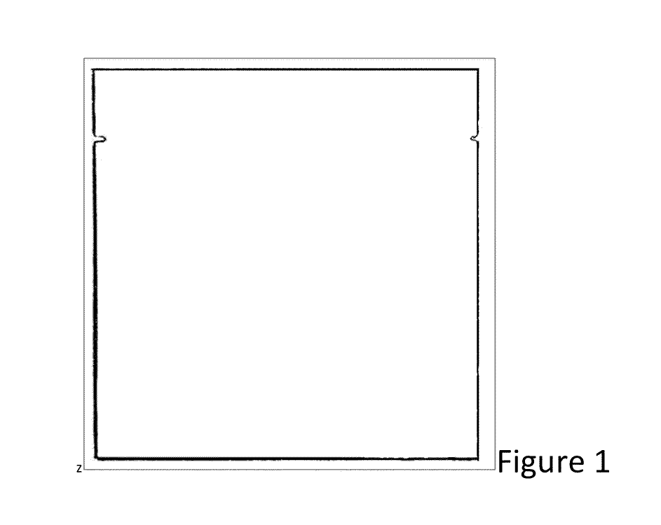

### DETAILED DESCRIPTION OF THE INVENTION

Skin brightener and skin discoloration products have been in the market for some time. In the last two decades, products such as skin discoloration fade cream and skin brightener cream and lotion are commonly found in pharmacies, make-up stores and department stores. What has not been created is the product at hand. There is a need for a portable medicated illuminating skin brightener wipe.

These wipes have all the benefits of portability without the downside of bottles and tubes. Cosmetics cream or gel can dehydrate and/or become contaminated and/or lose its potency over time. When a bottle is used, it is exposed to air and bacteria. When the user approaches the end of a bottle, the bottle has been used and exposed countless times. In applying the medicine the user must “eyeball” the amount that is to be applied, thereby applying either too much or little of the medicine. The creams must also be kept at proper temperatures ranging from 32° F. to 120° F., which prevent storage and use problems in regards to portability and eco friendly waste.

The practicality of this invention is quite simple. Instead of using a tube or jar of cream which is quite large, not as portable and does not maintain potency, this invention packages the wipes in a thin enclosed package this is portable, travel friendly, airport safe and stable for at least 5 years. The substrate used in the present invention comprises synthetic fiber in a non-woven, woven of knitted form. Of particular interest for use in the substrate employed in the present invention are the following fibers: polypropylene, polyester and rayon.

The skin brightener main composition, hydroquinone, is found in a number of patented skin brightener patents and products. Hydroquinone is used as a topical application in skin whitening to reduce the color of skin as it does not have the same predisposition to cause dermatitis as metol does

The other components that make up the composition currently in the marketplace includes: Aloe Vera, Bisabolol, Allantoin, Sodium Metabisulfate, Glycerin, Water, Propylene Glycol, Methylparaben and Propylparaben. While Hydroquinone is commonly found, the additional components are important for the solubility, consistency of the solution and ability to bind and preserve the substrate.

The use of propylene glycol with a small amount of water as a solvent is essential. Propylene glycol does not precipitate the release of free iodine, and thereby depletes its effectiveness before its actual use. Propylene glycol, unlike water, actually preserves the stability of the active and inactive ingredients and facilitates an extended shelf life of the treated towelette/wipe also known as substrate. Propylene glycol is a lubricious emollient imparting soothing and softening qualities of the skin. Propylene glycol is considered one of the most common moisture-carrying vehicles in cosmetic and OTC drug products second only to water. Propylene glycol does not freeze in cold weather reinforcing the stability of the overall product and negates the formation of any crystallization. The use of propylene glycol, as a non-aqueous solvent obviates the need for buffers, stabilizers, and preservatives which are generally required to be used in aqueous solutions.

Glycerin aids in moisturizing and coating the injured skin keeping it hydrated while it heals. Glycerin further helps to restore the healthy look and feel to the skin without introducing water which may dehydrate too quickly and not give the pain healing ingredients time to penetrate.

Aloe Vera for centuries has been known for its natural skin healing properties. It is known for naturally soothing, promoting healing and cellular renewal.

Methylparaben and Propylparabens aid in preserving the substrate.

The package of the wetted wipe is a laminate film that creates a barrier vehicle whose properties not only contains the medicated solution and substrate but prevents evaporation and contamination. Suitable and stable barrier materials are laminates, where a layer of ethylene vinyl alcohol or propylene vinyl alcohol or aluminum is sandwiched between polymeric film layers, such as low density polyethylene. Further suitable barrier materials for forming the package for the cleaning wipe herein are metallized, particularly aluminized plastics films; metal foils; oriented polyethylene terephtalate (PET); glycolmodified PET; oriented polyamidel; or polymeric films. Basically every material known in the art for this purpose is suitable for use herein. A very suitable laminate material for this purpose is for instance disclosed in EP 696,991.

The package of the wipes comprises opening aids for facilitating opening of the package. Exemplary opening aids are tear tapes, cuts or the like.

What is claimed is:

1. A packaged illuminating skin brightener wipe, comprising of
A disposable, flexible substrate suitable in size and strength for single-use application of a skin lightening medication solution; and a medicated illuminating skin brightener solution impregnated in said disposable, absorbent sheet material for providing over the counter strength skin brightener/lightener to said sheet material; and,
A hermetically sealed envelope retaining said disposable, absorbent sheet-material.

2. A packaged illuminating skin brightener wipe according to claim 1, wherein said hermetically sealed package is a tear-open packet.

3. A medicated illuminating skin brightener solution according to claim 1, wherein the composition consist of Hydroquinone.

4. A medicated illuminating skin brightener solution according to claim 1, wherein the solution also contains a combination of propylene glycol, glycerin and other additives to help with solubility, consistency of the solution and ability to bind and preserve the substrate.
